(19) United States
(12) Patent Application Publication
(10) Pub. No.: US 2008/0292696 A1
(43) Pub. Date: Nov. 27, 2008

(54) Enteric Sustained-Release Tablet Comprising Paroxetine

(76) Inventors: Sang Min Kim, Gyeonggi-do (KR); Woo Heon Song, Gyeonggi-do (KR)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(21) Appl. No.: 10/598,122

(22) PCT Filed: Apr. 28, 2006
(86) PCT No.: PCT/KR2006/001598

§ 371 Date: Aug. 18, 2006

(30) Foreign Application Priority Data
Nov. 4, 2005 (KR) 1020050105383

(51) Int. Cl. A61K 9/24; A61K 31/4525

(52) U.S. Cl. 424/465; 424/472; 514/321

(57) ABSTRACT

The present invention relates to an enteric, sustained-release tablet comprising paroxetine or a hydrates or anhydrides of a pharmaceutically acceptable salt thereof as active substance, more particularly to a tablet prepared by coating a sustained-release tablet core containing paroxetine with an enteric polymer, wherein the interaction between the tablet core and the enteric coating layer is minimized to enable constant drug release without regard to the residence time of the tablet in the stomach.

### TECHNICAL FIELD

The present invention relates to an enteric, sustained-release tablet comprising paroxetine or hydrates or anhydrides of a pharmaceutically acceptable salt thereof (hereunder collectively referred to as paroxetine) as active substance, more particularly to a tablet prepared by coating a sustained-release tablet core containing paroxetine with an enteric polymer, wherein the interaction between the tablet core and the enteric coating layer is minimized to enable constant drug release without regard to the residence time of the tablet in the stomach.

### BACKGROUND ART

A sustained-release dosage form is a dosage form designed to maintain the optimum blood level of a drug by controlling its release at a predetermined rate. The general purposes of sustained-release dosage forms are, by constantly maintaining the blood level of the drugs within an effective blood level range, to reduce the number of administrations, thereby improving patient compliance, and to reduce adverse drug reactions. Various sustained-release dosage forms have been developed to attain the objectives, which are, to attain the sustained release of drugs for the improvement of therapeutic effect and a reduction of adverse reactions. For example, use of a mechanical or osmotic pump (U.S. Pat. No. 4,765,989), a membrane-assisted diffusion control, a capsule formulation comprising a drug-containing core coated with a membrane, a matrix formulation in which a drug is dispersed in a drug release control layer, etc. have been proposed. U.S. Pat. No. 6,548,084 discloses an enteric-coated bilayer formulation comprising a matrix layer and a support layer for preventing excessive release of drug at early stage.

Paroxetine is a selective serotonin reuptake inhibitor (SSRI) effective in preventing or treating depression. For reasons of stability, it is generally used in the form of a pharmaceutical acceptable salt, typically paroxetine hydrochloride hemihydrate.

Immediate-release paroxetine drugs are known to cause adverse gastrointestinal reactions such as nausea, vomiting, etc (DeVane C L. Comparative safety and tolerability of selective serotonin reuptake inhibitors. Hum. Psychopharmacol. 1995; 10(suppl.): 185-193). It is reported that such adverse reactions are mainly caused by abrupt increases in the blood level of the drug and differences in the highest and lowest blood levels. Also, it is known that 5-HT3 and 5-HT receptor subtypes, which are present mainly in the upper gastrointestinal tract, cause the adverse reactions (Leatherman M E, Bebchuk J M, Ekstrom R D, Heine A D, Carson S W, Golden R N. The effects of serotonin3 receptor blockade on the psychobiological response to intravenous clomipramine in healthy human subjects. Biol. Psychiarty: 1999; 45:238-240). Considering that an antidepressant has to be taken for a long time, it is important to improve patient compliance by making it convenient to take and reduce adverse gastrointestinal reactions such as nausea and vomiting. To do so, the drug should not be released while residing in the stomach, i.e., it should be released only after it reaches the small intestine. Second, drug release in the small intestine should be performed at a constant rate as originally designed.

The technique of coating an oral drug with an enteric polymer with the purpose of preventing it from being released in the stomach, i.e., preventing it from being released in the acidic pH of the stomach and inducing it to be released in the neutral pH of the small intestine, is well known in the art.

However, according to the finding of the present inventors, when an enteric coating layer is directly coated on a sustained-release tablet core comprising paroxetine, the release behavior of the tablet changes significantly because of the interaction between the tablet core and the enteric coating layer. That is, when a directly enteric-coated sustained-release tablet comprising paroxetine is transferred from the acidic environment of the stomach to the neutral pH of the small intestine, the release rate changes significantly, without being maintained as intended. The change in release rate becomes increasingly severe the longer the drug is exposed to the acidic pH. Such change in drug release behavior may cause a severe problem, considering that the gastric emptying time, or the time required for an orally administered drug to be transferred from the stomach to the small intestine, varies a lot inter and intraindividually. In other words, a directly enteric-coated matrix type sustained-release tablet comprising paroxetine may not offer consistent therapeutic effect because release rate or release time of the paroxetine changes every time the drug is administered, thereby causing significant change in the amount of drug uptake.

To conclude, a paroxetine-containing dosage form intended for oral administration has to be an enteric-coated sustained-release dosage form designed to minimize adverse reactions, offer consistent therapeutic effect and maintain drug release rate regardless of the residence time of the tablet in the stomach.

### DISCLOSURE OF INVENTION

### Technical Solution

The present inventors worked to develop a sustained-release tablet comprising paroxetine, that minimizes the interaction between the sustained-release tablet core and the enteric coating layer and maintains the drug release rate without regard to the residence time in the stomach. In doing so, the present inventors found out that a proper separation layer introduced between the tablet core and the enteric coating layer offers a solution.

One of the objectives of the present invention is to provide an enteric, sustained-release tablet comprising paroxetine as active substance and a method for preparing the same, more particularly to provide an enteric, sustained-release tablet comprising paroxetine, in which the interaction between the tablet core and the enteric coating layer can be minimized, enabling a constant release rate or release time without regard to the residence time of the drug in the stomach.

In order to attain this objective, the present invention provides an enteric, sustained-release tablet comprising paroxetine, which comprises: a tablet core prepared by preparing granules comprising paroxetine and high-viscosity and low-viscosity hydroxypropylmethylcellulose and further adding low-viscosity hydroxypropylmethylcellulose to the granules and compressing; a separation layer that separates the tablet core from the enteric coating layer; and an enteric coating layer.

The present invention is characterized by the preparation of granules comprising paroxetine and high-viscosity and low-viscosity hydroxypropylmethylcellulose and making them into a tablet, so designed to enable the sustained release of the drug.

The present invention is also characterized by the coating of the drug with an enteric polymer, so that the drug is released after it reaches the small intestine.

The present invention is further characterized by the introduction of a separation layer between the tablet core and the enteric coating layer to minimize the interaction between them.

Hereunder is given a more detailed description of the present invention.

The paroxetine referred in the present invention encompasses paroxetine and any pharmaceutically acceptable salt thereof, including hydrates or anhydrides; typically, paroxetine hydrochloride hemihydrate. The paroxetine-containing tablet core used in the present invention is a matrix type sustained-release core. It comprises hydroxypropylmethylcellulose for sustained release of the drug. More particularly, it is prepared by preparing granules comprising paroxetine and high-viscosity and low-viscosity hydroxypropylmethylcellulose and further adding low-viscosity hydroxypropylmethylcellulose.

The viscosity of hydroxypropylmethylcellulose refers to one in a 2% aqueous solution (20° C.). The low-viscosity hydroxypropylmethylcellulose refers to one having a viscosity ranging from 40 to 60 cps and the high-viscosity hydroxypropylmethylcellulose refers to one having a viscosity ranging from 3,000 to 14,000 cps.

The high-viscosity hydroxypropylmethylcellulose is comprised in the paroxetine-containing granules within from 3 to 30 w/w %, preferably within from 3 to 20 w/w %. And, the low-viscosity hydroxypropylmethylcellulose is comprised in the granules within from 10 to 40 w/w %, preferably within from 10 to 30 w/w %.

The paroxetine-containing granules are comprised in the tablet core within from 40 to 90 w/w %, preferably within from 60 to 90 w/w %.

To the resulting paroxetine-containing granules, low-viscosity hydroxypropylmethylcellulose may be further added to prepare the tablet core. In this case, the low-viscosity hydroxypropylmethylcellulose is added within from 10 w/w % to 40 w/w %, preferably within from 10 w/w % to 30 w/w %, based on the total weight of the tablet core.

During the preparation of the paroxetine-containing granules and subsequent addition of the low-viscosity hydroxypropylmethylcellulose, pharmaceutically acceptable excipients, binders (e.g., polyvinylpyrrolidone, hydroxypropylcellulose, methylcellulose, etc.), lubricants (e.g., glyceryl behenate, light anhydrous silicic acid, magnesium stearate), disintegrants (e.g., sodium starch glycolate, croscarmellose sodium, crospovidone, etc.), etc. may also be added.

For the excipients, it is preferable if at least one is selected from the group consisting of lactose, microcrystalline cellulose, starch, mannitol and calcium hydrogen phosphate. Among these, either lactose or microcrystalline cellulose is the most preferable.

The present invention is also characterized by the separation layer which exists between the tablet core and the enteric coating layer.

The present inventors found that, for unknown reason when an enteric coating layer is introduced directly to the paroxetine-containing tablet core, the drug release behavior of the tablet changes significantly because of the interaction between the tablet core and the enteric coating layer. Further, the present inventors found that this phenomenon becomes severe when the tablet resides in the low-pH environment of the stomach for a length of time. Such a change in drug release behavior has to be solved because it may interrupt consistent therapeutic effect even intraindividually. The present inventors found out that this problem can be solved completely if the proper separation layer is introduced between the tablet core and the enteric coating layer.

The separation layer is formed using at least one polymer, either water-soluable or in-soluable, selected from the following groups.

The water-insoluable group comprise: ethylcellulose [e.g., Surelease (Colorcon); Aquacoat ECD (FMC), etc.], polyvinylacetate [e.g., Kolllicoat SR (BASF)], ammoniomethacrylate copolymer type B [e.g., Eudragit RS (Degussa)], etc. Among these, ethylcellulose is the most preferable.

The water-soluable group comprise: hydroxypropylmethylcellulose, methylcellulose, polyvinylpyrrolidone, hydroxypropylcellulose, ammoniomethacrylate copolymer type A [e.g.,: Eudragit RL (Degussa)], polyvinylalcohol, etc. Among these, hydroxypropylmethylcellulose is the most preferable. Preferably, hydroxypropylmethylcellulose used for this purpose has a viscosity ranging from 2 to 20 cps.

The separation layer may further comprise a pharmaceutically acceptable plasticizer (e.g., medium chain triglyceride, triethylcitrate, propylene glycol, etc.), a lubricant, and/or a light stabilizer (e.g., TiO2, etc.), etc.

Preferably, the separation layer is comprised within from 1 to 30 w/w %; more preferably from 3 to 15 w/w %, based on the weight of the tablet core.

The present invention is also characterized by the enteric coating layer which is additionally formed on the outside of the tablet wherein the said separation layer has been applied.

In the present invention, the tablet is prepared by coating the tablet with an enteric coating material in order to prevent the drug from being released in the stomach and instead induce it to be released in the small intestine.

The enteric coating layer may be prepared from a pharmaceutically acceptable enteric polymer, for example, methacrylate copolymer [e.g., Acryleze (Colorcon); Eudragit L100 or L-100-55 (Degussa), etc.], hydroxypropylmethylcellulose phthalate), hydroxypropylmethylcellulose acetate phthalate), cellulose acetate phthalate), carboxymethylethylcellulose, etc.

The enteric coating layer may further comprise a pharmaceutically acceptable plasticizer (e.g., medium chain triglyceride, triethylcitrate, propylene glycol, etc.), a lubricant, and/or a light stabilizer (e.g., TiO2, etc.).

Preferably, the enteric coating layer is comprised within from 5 to 20 w/w %, based on the weight of the tablet wherein the said separation layer has been applied.

The present invention also provides a method for preparing an enteric, sustained-release tablet comprising paroxetine. The steps involve: 1) preparing granules comprising paroxetine and high-viscosity and low-viscosity hydroxypropylmethylcellulose; 2) preparing a tablet core by adding low-viscosity hydroxypropylmethylcellulose to the granules and compressing them; 3) introducing a separation layer to the tablet core; and 4) introducing an enteric coating layer to the tablet wherein the said separation layer has been applied.

1) Preparation of Granules Comprising Paroxetine and High-Viscosity and Low-Viscosity Hydroxypropylmethylcellulose

Paroxetine is mixed with high-viscosity and low-viscosity hydroxypropylmethylcellulose. A solvent is added and the resulting mixture is granulated.

The paroxetine used in the present invention encompasses paroxetine and pharmaceutically acceptable salts thereof, including hydrates or anhydrides; typically, paroxetine hydrochloride hemihydrate.

The high-viscosity hydroxypropylmethylcellulose refers to one having a viscosity ranging from 3,000 to 14,000 cps. The low-viscosity hydroxypropylmethylcellulose refers to one having a viscosity ranging from 40 to 60 cps.

The solvent used in the present invention may be water, ethanol, isopropanol, methylene chloride, acetone, etc. or a mixture thereof. In actual fact, any pharmaceutical acceptable solvent capable of dispersing or dissolving the drug and the hydroxypropylmethylcellulose may be used, without being limited to the afore-mentioned examples.

The paroxetine-containing granules may further comprise pharmaceutically acceptable excipients, binders (e.g., polyvinylpyrrolidone, hydroxypropylcellulose, methylcellulose, etc.), lubricants (e.g., glyceryl behenate, light anhydrous silicic acid, magnesium stearate), disintegrants (e.g., sodium starch glycolate, croscarmellose sodium, crospovidone, etc.), etc.

For the excipient, at least one selected from the group consisting of lactose, microcrystalline cellulose, starch, mannitol and calcium hydrogen phosphate is preferable. Among these, either lactose or microcrystalline cellulose is the most preferable.

The granules are prepared by the commonly used method. That is, mechanical appliances such as screw type extrusion granulators, cylindrical granulators, oscillating granulators, planetary mixers, vertical granulators [Hi-speed mixer (Freund)], etc., may be used. The resulting granulates are dried and screened to an adequate size to prepare the granules.

2) Preparation of the Tablet Core by Adding Low-Viscosity Hydroxypropylmethylcellulose

Low-viscosity hydroxypropylmethylcellulose is then added to the paroxetine-containing granules and then the mixture is compressed to form the tablet core.

During the mixing, pharmaceutically acceptable excipients, binders (e.g., polyvinylpyrrolidone, hydroxypropylcellulose, methylcellulose, etc.), lubricants (e.g., glyceryl behenate, light anhydrous silicic acid, magnesium stearate), disintegrants (e.g., sodium starch glycolate, croscarmellose sodium, crospovidone, etc.), etc. may be added.

The compression may be performed using a commonly used rotary press, etc.

3) Introduction of the Separation Layer

A separation layer, comprising at least one selected from the group consisting of water-insoluble and water-soluble polymers, is introduced to the tablet core in order to separate the paroxetine-containing tablet core from the enteric coating layer. For the water-insoluble polymer, ethylcellulose [e.g., Surelease (Colorcon); Aquacoat ECD (FMC), etc.], polyvinylacetate (e.g., Kolllicoat SR (BASF)), ammoniomethacrylate copolymer type B [e.g., Eudragit RS (Degussa)], etc. may be used. Among them, ethylcellulose is the most preferable.

For the water-soluble polymer, hydroxypropylmethylcellulose, methylcellulose, polyvinylpyrrolidone, hydroxypropylcellulose, ammoniomethacrylate copolymer type A [e.g., Eudragit RL (Degussa)], polyvinylalcohol, etc. may be used. Among them, hydroxypropylmethylcellulose is the most preferable. Preferably, the hydroxypropylmethylcellulose used for this purpose has a viscosity ranging from 2 to 20.

The polymer may be prepared into a composition for forming the separation layer by dissolving or dispersing in water or an organic solvent. The organic solvent may be ethanol, isopropanol, methylene chloride, acetone, etc. or any mixture thereof and is not limited to the afore-mentioned examples.

The composition for forming the separation layer may comprise pharmaceutically acceptable plasticizers (e.g., medium chain triglyceride, triethylcitrate, propylene glycol, etc.), lubricants, light stabilizers (e.g., TiO2, etc.), etc.

4) Introduction of the Enteric Coating Layer

The enteric coating layer may be prepared from an enteric polymer, such as methacrylate copolymer [e.g., Acryleze (Colorcon); Eudragit L100 or L-100-55 (Degussa), etc.], hydroxypropylmethylcellulose phthalate, hydroxypropylmethylcellulose acetate phthalate, cellulose acetate phthalate, carboxymethylethylcellulose, etc. In addition to the enteric polymer, pharmaceutically acceptable plasticizers (e.g., medium chain triglyceride, triethylcitrate, propylene glycol, etc.), lubricants, light stabilizer (e.g., TiO2, etc.), etc. may be added.

The application of the separation layer and the enteric coating layer may be performed with a commonly used coating machine, such as a side-vented coater [e.g. Hi-Coater, (Freund)], a perforated coater [e.g., Accela-Cota (Thomas Engineering), etc.

### BRIEF DESCRIPTION OF THE DRAWINGS

FIG. 1 shows the drug release test result for Examples 1 to 4.

FIG. 2 shows the drug release test result for Comparative Example 1.

### BEST MODE FOR CARRYING OUT THE INVENTION

The most practical and preferred embodiments of the present invention are illustrated in the following examples. However, it is understood that those skilled in the art, in consideration of this disclosure, may make modifications and improvements within the spirit and scope of the present invention.

### Examples 1 and 2

80 g ethanol was added to a mixture of paroxetine hydrochloride hemihydrate, lactose, microcrystalline cellulose and low-viscosity and high-viscosity hydroxypropylmethylcellulose (see Table 1). The mixture was granulated with a planetary mixer, dried and screened to granules. Low-viscosity hydroxypropylmethylcellulose, light anhydrous silicic acid, glyceryl behenate and magnesium stearate was then added to the resulting granules. The mixture was compressed and formed into a round-shape tablet core. The tablet core was coated with a separation layer (See table 1) and then an enteric coating layer. The composition for forming the separation layer was prepared by completely dissolving hydroxypropylmethylcellulose and polyethylene glycol in water and then dispersing an ethylcellulose aqueous dispersion (Surelease™). The enteric coating solution was prepared by completely dispersing a methacrylate copolymer mixture (Acryleze™) in water. The composition for forming the separation layer and the enteric coating solution was coated on the tablet core using Hi-Coater to obtain an enteric, sustained-release tablet comprising paroxetine.

### Example 3

A separation layer was introduced then coated with an enteric coating layer in the same manner as in Example 1. The composition for forming the separation layer was prepared by completely dissolving hydroxypropylmethylcellulose and polyethylene glycol in water. An enteric, sustained-release tablet comprising paroxetine was prepared in the same manner as in Examples 1 and 2.

### Example 4

A separation layer and was introduced then coated with an enteric coating layer in the same manner as in Example 1. The composition for forming the separation layer was prepared by completely dispersing an ethylcellulose aqueous dispersion (Surelease™) in water. An enteric, sustained-release tablet comprising paroxetine was prepared in the same manner as in Examples 1 and 2.

### Comparative Example 1

An enteric, sustained-release tablet comprising paroxetine was prepared in the same manner as in Example 1, except that a separation layer was not introduced.

TABLE 1
(Unit: g, HPMC: hydroxypropylmethylcellulose)

| | | | | | | Comp. |
|---|---|---|---|---|---|---|
| | Composition | Ex. 1 | Ex. 2 | Ex. 3 | Ex. 4 | Ex. 1 |
| Preparation of | Paroxetine hydrochloride | 14.25 | 14.25 | 14.25 | 14.25 | 14.25 |
| granules | hemihydrate | | | | | |
| | HPMC (10000 cps) | 9 | — | 9 | 9 | 9 |
| | HPMC (4000 cps) | — | 9 | — | — | — |
| | HPMC (50 cps) | 27 | 27 | 27 | 27 | 27 |
| | Lactose | 72.85 | 72.85 | 72.85 | 72.85 | 72.85 |
| | Microcrystalline cellulose | 12.15 | 12.15 | 12.15 | 12.15 | 12.15 |
| Post-mixing | HPMC (50 cps) | 27 | 27 | 27 | 27 | 27 |
| | Glyceryl behenate | 9 | 9 | 9 | 9 | 9 |
| | Light anhydrous silicic acid | 1 | 1 | 1 | 1 | 1 |
| | Magnesium stearate | 1 | 1 | 1 | 1 | 1 |
| Introduction of | Ethylcellulose (Surelease) | 2.55 | 2.55 | — | 5 | — |
| separationlayer | HPMC (5 cps) | 10.21 | 10.21 | 13.64 | — | — |
| | Talc | 1.22 | 1.22 | — | 0.5 | — |
| | Polyethylene glycol | 1.02 | 1.02 | 1.36 | — | — |
| Enteric coating | Methacrylate copolymer | 15 | 15 | 15 | 15 | 15 |
| | mixture(Acryleze ™) | | | | | |

### Testing Example

Drug release tests were performed for the tablets prepared in Examples 1 to 4 and Comparative Example 1.

Drug Release Test 1 (DRT 1)

The drug release test was performed in 1000 mL of a pH 7.5 tris buffer solution at 150 rpm, in accordance with KP (Korean Pharmacopoeia) Dissolution Test Method No. 2.

Drug Release Test 2 (DRT 2)

After performing the drug release test for 2 hours in 750 mL of 0.1 N HCl at 150 rpm, in accordance with KP Dissolution Test Method No. 2, to simulate the acidic environment, the drug release test was subsequently performed in 1000 mL of a pH 7.5 tris buffer solution in the same manner as in Drug release Test 1.

TABLE 2
Accumulative drug release rate (%)

| | | | | | | | | | Comparative | |
|---|---|---|---|---|---|---|---|---|---|---|
| | Example 1 | | Example 2 | | Example 3 | | Example 4 | | Example 1 | |
| Time | DRT | DRT | DRT | DRT | DRT | DRT | DRT | DRT | DRT | DRT |
| (hr) | 1 | 2* | 1 | 2 | 1 | 2 | 1 | 2 | 1 | 2 |
| 0.25 | 0 | 0 | 0 | 0 | 1.90 | 0 | 0 | 0 | 1.53 | 0 |
| 0.5 | 2.34 | 0.84 | 2.21 | 1.55 | 9.61 | 3.77 | 0 | 0.07 | 3.83 | 0.44 |
| 1 | 14.50 | 11.92 | 13.67 | 11.44 | 23.85 | 17.54 | 0.75 | 0.81 | 9.88 | 4.13 |
| 1.5 | 28.15 | 26.50 | 25.51 | 23.71 | 36.35 | 30.60 | 2.51 | 2.49 | 17.13 | 8.52 |
| 2 | 41.45 | 39.96 | 36.59 | 35.40 | 47.31 | 42.24 | 5.60 | 5.64 | 26.81 | 13.36 |
| 3 | 62.96 | 60.84 | 57.41 | 55.98 | 65.25 | 61.84 | 14.97 | 15.63 | 46.05 | 24.37 |
| 4 | 78.76 | 75.62 | 74.01 | 72.42 | 79.87 | 77.53 | 31.10 | 30.06 | 68.09 | 40.74 |
| 5 | 90.37 | 86.90 | 86.23 | 84.58 | 90.82 | 89.26 | 47.23 | 46.35 | 86.34 | 61.15 |
| 6 | 96.89 | 94.37 | 94.34 | 92.89 | 97.71 | 96.58 | 62.31 | 61.29 | 98.45 | 79.68 |
| 8 | 97.79 | 98.39 | 100.12 | 98.09 | 100.55 | 99.53 | 90.12 | 88.06 | 103.32 | 96.88 |

(*DRT 2 is drug release rate of paroxetine after transition toin pH 7.5 tris buffer from 0.1N HCl was changed to 7.5)

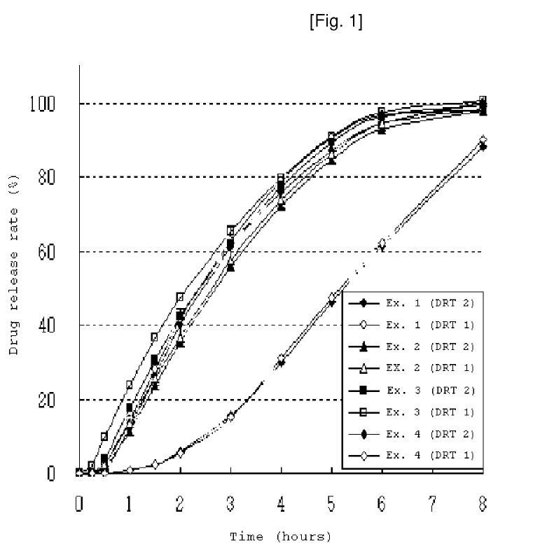

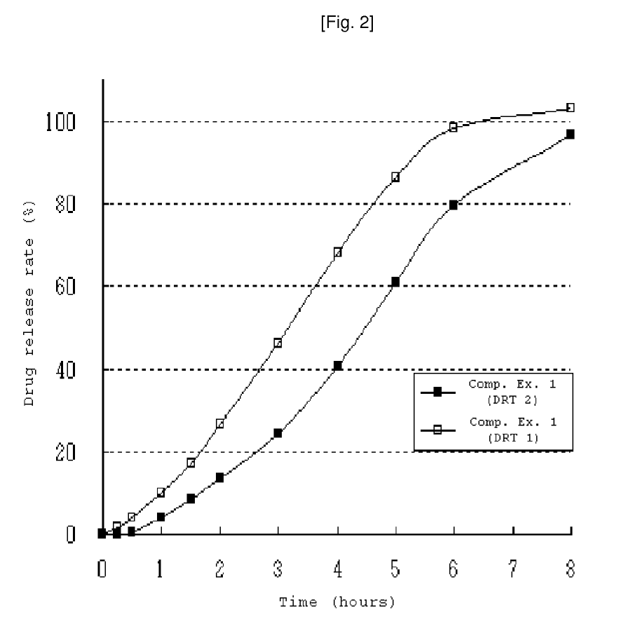

As seen in Table 2 and FIGS. 1 and 2, the drug release rate decreased significantly, when the dissolution testing was performed for 2 hours in an acidic condition followed by a neutral one, in Comparative Example 1. In contrast, Examples 1 to 4 maintained the original drug release behavior regardless of the change in buffer solutions. This shows that an orally-administered enteric, sustained-release tablet comprising paroxetine in accordance with the present invention can maintain the originally designed drug release behavior after being transferred to the small intestine, without regard to the residence time in the stomach.

### INDUSTRIAL APPLICABILITY

The enteric, sustained-release tablet comprising paroxetine, in accordance with the present invention, is capable of maintaining the drug release behavior without regard to the residence time in the stomach, as well as reducing gastrointestinal adverse reactions such as nausea, vomiting, etc. characteristic of immediate-release preparations comprising paroxetine, as the interaction between the paroxetine-containing sustained tablet core and the enteric coating layer is minimized. Therefore, the present invention provides a proper paroxetine preparation capable of reducing adverse reactions and reducing variations in therapeutic effect inter and intraindividually, thereby improving patient compliance.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the present invention as set forth in the appended claims.

1. An enteric, sustained-release tablet comprising paroxetine in which a separation layer is introduced between a sustained-release tablet core comprising paroxetine as pharmaceutically active substance and an enteric coating layer enclosing the tablet core in order to maintain the drug release behavior without regard to the residence time of the drug in an acidic environment, wherein the separation layer is prepared from a mixture of a) at least one water-insoluble polymer selected from the group consisting of ethylcellulose, polyvinylacetate and ammoniomethacrylate copolymer type B and b) at least one water-soluble polymer selected from the group consisting of hydroxypropylmethylcellulose, methylcellulose, polyvinylpyrrolidone, hydroxypropylcellulose, ammoniomethacrylate copolymer type A and polyvinylalcohol and completely encloses the tablet core.

2. An enteric, sustained-release tablet comprising paroxetine in which a separation layer is introduced between a sustained-release tablet core comprising paroxetine as pharmaceutically active substance and an enteric coating layer enclosing the tablet core in order to maintain the drug release behavior without regard to the residence time of the drug in an acidic environment, wherein the separation layer is prepared from a water-soluble polymer selected from the group consisting of hydroxypropylmethylcellulose, methylcellulose, polyvinylpyrrolidone, hydroxypropylcellulose, ammoniomethacrylate copolymer type A and polyvinylalcohol and completely encloses the tablet core.

3. The enteric, sustained-release tablet comprising paroxetine as set forth in claim 1 or claim 2, wherein the paroxetine is paroxetine hydrochloride hemihydrate.

4. The enteric, sustained-release tablet comprising paroxetine as set forth in claim 1 or claim 2, wherein the separation layer is comprised within from 1 to 30 w/w % based on the weight of the tablet core.

5. The enteric, sustained-release tablet comprising paroxetine as set forth in claim 1 or claim 2, wherein the tablet core is prepared by further adding low-viscosity hydroxypropylmethylcellulose to granules comprising paroxetine and high-viscosity and low-viscosity hydroxypropylmethylcellulose.

6. The enteric, sustained-release tablet comprising paroxetine as set forth in claim 5, wherein the high-viscosity hydroxypropylmethylcellulose has a viscosity ranging from 3,000 to 14,000 cps and the low-viscosity hydroxypropylmethylcellulose has a viscosity ranging from 40 to 60 cps.

7. The enteric, sustained-release tablet comprising paroxetine as set forth in claim 5, wherein the paroxetine-containing granules are comprised in the tablet core within from 40 to 90 w/w % based on the total weight of the tablet core.

8. The enteric, sustained-release tablet comprising paroxetine as set forth in claim 5, wherein the paroxetine-containing granules comprise 3 to 30 w/w % of high-viscosity hydroxypropylmethylcellulose and 10 to 40 w/w % of low-viscosity hydroxypropylmethylcellulose.

9. The enteric, sustained-release tablet comprising paroxetine as set forth in claim 1, wherein the enteric coating layer is prepared from a material selected from the group consisting of methacrylate copolymer, hydroxypropylmethylcellulose phthalate, hydroxypropylmethylcellulose acetate phthalate, cellulose acetate phthalate and carboxymethylethylcellulose.

10. The enteric, sustained-release tablet comprising paroxetine as set forth in claim 5, which further comprises pharmaceutically acceptable excipients, binders, lubricants, disintegrants, etc. in the tablet core.
